(19) United States
(12) Patent Application Publication
(10) Pub. No.: US 2003/0138965 A1
(43) Pub. Date: Jul. 24, 2003

(54) Systems and methods for measuring nitrate levels

(76) Inventors: George A. Allen, Swampscott, MA (US); Petros Koutrakis, Weston, MA (US); Yiming Ding, Quincy, MA (US)

Correspondence Address: Chinh H. Pham, PATENT DEPARTMENT, FOLEY HOAG LLP, 155 Seaport Boulevard, Boston, MA, 02210, US

(21) Appl. No.: 10/265,889

(22) Filed: Oct. 7, 2002

Related U.S. Application Data
(63) Continuation-in-part of application No. 09/687,190, filed on Oct. 12, 2000, now Pat. No. 6,503,758.
(60) Provisional application No. 60/158,861, filed on Oct. 12, 1999.

(51) Int. Cl. G01N 33/00

(52) U.S. Cl. 436/110; 422/88

(57) ABSTRACT

The present disclosure is directed to embodiments of systems for measuring nitrate levels. The systems disclosed herein may include a sample inlet to permit introduction of a gas sample from which nitrate levels may be measured and a filter downstream from the sample inlet to trap particles. A first heating zone to volatilize the trapped particles may be coupled to the filter. Situated between the filter and the sample inlet may be a gas inlet to provide a secondary stream of gas to be passed over the trapped particles to minimize oxidation of low nitrogen containing compounds during heating. Downstream from the filter may be a second heating zone to heat a pathway adjacent to the filter to minimize particles depositing on internal surfaces of the pathway, and downstream from the second heating zone may be an analyzer to measure nitrate levels of the gas sample downstream from the filter. Also disclosed herein are methods for measuring nitrate levels.

The present application is a continuation-in-part of U.S. Application No. 09/687,190, filed Oct. 12, 2000, which claims priority to U.S. Provisional Application No. 60/158,861, filed Oct. 12, 1999. Each of the above-referenced applications is incorporated herein by reference.

### BACKGROUND OF THE INVENTION

Particulates are tiny clumps of soot, dirt, and various chemicals that have been linked to a wide variety of health problems—e.g., asthma and higher rates of disease affecting the cardiovascular system or lungs. Since 1987, EPA standards have governed all particultes under 10 micrometers in diameter. This category of particulate matter is called PM10. Recently, however, studies have suggested that the most dangerous particles are actually the smaller ones, which penetrate deeper in the lungs' aereoles. Thus, new regulations will build in a separate standard for particles less than 2.5 micrometers in diameter—PM2.5.

While PM10 contains a lot of wind-blown soil, PM2.5 is derived mainly from burning fossil fuels. PM2.5 typically contains a mixture of elemental carbon, organic carbon, sulfate and nitrate particles, and acid droplets. It is unlikely that all components of PM2.5 contribute equally to the observed health effects, yet the present lack of sufficient data quantifying the individual components prevents the EPA from separately regulating these components. Because regulating PM2.5 collectively is not a cost-effective solution, the agency is under great scientific, industrial, and political pressure to specifically identify sources of the observed particle health-effects. Thus, interest in measuring the individual components of PM2.5 has increased dramatically over the last few years.

A number of methods are known for measuring atmospheric nitrate levels. Koutrakis et al., Environ. Sci. Technol. 22:1463, 1988 disclose an integrated sampling method (Harvard/EPA Annular Denuder System (HEADS)) which is designed to measure various atmospheric components including particulate nitrate. The method provides a non-quantitative conversion of particles nitrate to nitric acid vapor by collection of atmospheric fine particles on a TEFLON® filter, with a sodium carbonate-coated filter downstream to collect nitric acid vapor produced by volatization of ammonium nitrate and by the reaction of ammonium nitrate with acidic sulfate particles.

Wendt et al., “Continuous monitoring of atmospheric nitric oxide and nitrogen dioxide by chemiluminescence” in Methods of Air Sampling and Analysis, editor, J. P. Lodge Jr., Lewis Publishers, Chelsea, Mich., pp 415-421 (1989), disclose a continuous chemiluminescent NOₓ detection method. Yamamoto et al., Anal. Chem., 1994, 66, 362-367, describe a nitrate analysis method relying on chemiluminescent NOₓ detection. NOₓ generally refers to NO₂ and NO taken together.

Brauer et al., Environ. Sci. Technol. 24:1521, 1990 disclose a method for the continuous measurement of nitrous acid and nitric acid vapors which does not distinguish between the two species. Klockow et al., Atmospheric Environment, 1989, 23, 1131-1138, disclose thermodenuder systems for the discontinuous measurement of nitric acid vapor and ammonium nitrate. Buhr et al., Atmospheric Environment, 1995, 29, 2609-2624, teach a denuder for sampling nitric acid, nitrate, and sulfate. Wolfson et al., U.S. Pat. No. 5,854,077, present a continuous differential nitrate measurement method.

Many of these and other existing methods for nitrate measurement require labor-intensive, manual collection of 24-hour integrated samples and laboratory analysis of the collected components. Not only are such samples expensive to collect, but the lengthy collection period prevents the detection of cycles and patterns which occur over the course of a day. Convenient techniques which offer improved temporal resolution and are capable of unifying the collection and analysis processes are badly needed now to reveal these daily patterns, both for epidemiological research and for regulatory monitoring.

### SUMMARY OF THE INVENTION

The systems and methods described herein relate to the measurement of nitrate in gas samples by collection and analyzing samples by a technique which permits a short cycling time. Thus, in one aspect, the invention provides a system for measuring nitrate levels having a sample inlet for receiving a sample of gas, a collection body coupled to said sample inlet, a filter mounted within said body to collect particles from said sample of gas, a heater coupled to the body to heat the body, a gas inlet coupled to said body to provide a flow of gas through said body, and a detector coupled to said body to measure an NOₓ concentration.

In a certain embodiment, the system further comprises a source of gas coupled to said gas inlet. The gas may be nitrogen or another gas which is substantially free of oxygen.

In another embodiment, the system also includes a catalyst, coupled to said body and to said detector, capable of reducing NO₂ to NO. The catalyst may comprise molybdenum, carbon, or ferrous sulfate.

In certain embodiments, the detector included in the system has a light sensor, and may further include an ozone generator, for example, for the detection of the chemiluminescent oxidation of NO. In another embodiment, the detector includes an infrared sensor. In yet another embodiment, the detector includes a material which reversibly binds NO.

In one embodiment, the filter mounted within the body to collect particles from the gas sample comprises quartz fibers. In another embodiment, the filter comprises polytetrafluoroethylene (PTFE) fibers.

In yet another embodiment, the system includes an extractor coupled to the sample inlet and to the collection body to substantially remove NO₂ from the gas sample. The extractor may comprise a hydroxyl-bearing solvent and a base, e.g., glycerol and an organic base, e.g., an amine, such as triethanolamine.

In yet another embodiment, the system also includes a selection platform, situated between the sample inlet and the extractor, to substantially remove particles larger than about 2.5 microns. The selection platform may be a filter, an inertial impactor, or any other suitable device.

In one embodiment, the system further includes a cooling system to cool the collection body.

In accordance with one embodiment, a nitrate level measuring system may include a sample inlet to allow introduction of a sample of gas from which nitrate levels may be measured and a desorption zone positioned distal to the sample inlet. Within the desorption zone there may be a filter to trap particles, such as nitrate, and a first heating zone to heat the filter and volatilize the trapped particles. In addition, the system may include a gas inlet, to provide a stream of gas substantially free of oxygen to be passed over the trapped particles. A second heating zone may also be provided to heat a pathway leading from the desorption zone to minimize incompletely volatized particles depositing on the internal surfaces of the pathway. The system may further include an alalyzer to measure nitrate levels.

The present invention also provides methods for measuring a level of nitrate. In an embodiment, the method includes receiving a gas sample, collecting nitrate particles from the gas sample on a filter, passing a stream of gas substantially free of oxygen over the collected particles, volatilizing the collected particles by heating to generate NOₓ, and measuring a level of NOₓ.

In another embodiment, the method further includes substantially removing NO₂ prior to collecting nitrate particles, e.g., by passing the received sample over a hydroxyl-bearing solvent and a base, e.g., an organic base such as triethanolamine.

In another embodiment, the method further includes removing particles larger than about 2.5 microns from the received gas sample, e.g., by passing the received sample through an inertial impactor or by passing the received sample through a filter.

In one embodiment of the method, passing a stream of gas includes passing a stream of nitrogen over the collected particles. In yet another embodiment, the method further comprises reducing generated NO₂ to NO using a metal catalyst, e.g., by contacting the NO₂ with a molybdenum catalyst.

In certain embodiments, measuring a level of NOₓ includes reacting NO with ozone. In yet another embodiment, measuring a level of NOₓ includes detecting infrared absorption. In certain other embodiments, measuring a level of NOₓ includes adsorbing NOₓ on a conductive material.

In one embodiment, collecting nitrate particles comprises collecting nitrate particles on a filter comprising quartz fibers.

In another embodiment, volatilizing the collected particles includes rapidly heating the collected particles to at least 300° C.

In yet another aspect, the invention provides a system for measuring nitrate levels, including a sample inlet to receive a sample of gas, an extractor coupled to said sample inlet to substantially remove NO₂ from the gas sample, a collection body coupled to said sample inlet, an inertial impactor mounted within said body to collect particles from the gas sample, a current source coupled to the inertial impactor to heat the inertial impactor and generate NOₓ and a detector coupled to said catalyst to measure an NOₓ concentration.

In yet another aspect, the invention relates to a method for measuring a level of nitrate by receiving a gas sample, substantially removing NO₂ from the gas sample, collecting nitrate particles from the gas sample with an inertial impactor, passing a stream of gas substantially free of oxygen over the collected particles, volatilizing the collected particles by heating to generate NOₓ, and measuring a level of NOₓ generated by the heated particles.

In accordance with another embodiment, the method for measuring nitrate level may include introducing a sample of gas from which nitrate levels may be measured and trapping particles found within the sample of gas on a filter. Thereafter, a secondary stream of gas substantially free of oxygen may be passed over the particles trapped on the filter, and the filter may be heated to volatilize the trapped particles. Subsequently, the volatilized particles in a pathway carried downstream from the filter by the secondary stream of gas may be heated to minimize the deposition of the volatilized particles on the internal surfaces of the pathway. The level of nitrate may thereafter be measured.

In yet another aspect, the invention provides a system for measuring nitrate levels having means for receiving a sample of gas, support means coupled to the means for receiving, means for collecting particles coupled to the support means, means coupled to the support means for heating the support means to generate NOₓ, means coupled to the support means for flowing a stream of gas through the support means, and means for measuring an NOₓ concentration coupled to the support means.

The system may also include means for substantially removing NO₂ from the sample of gas, coupled to said means for receiving and said support means. Such a system may further include means for reducing NO₂ to NO, coupled to the support means and to the means for measuring.

In accordance with another embodiment, a system for measuring nitrate levels may include means for introducing a sample of gas from which nitrate levels may be measured into the system and means for trapping particles found within the sample of gas. Additionally, the system may include means for passing a stream of gas substantially free of oxygen over the trapped particles, means for heating the trapped particles to volatilize the trapped particles, means for heating the gas in a pathway beyond the means for trapping particles to minimize the volatilized particles depositing on internal surfaces of the pathway, and means for measuring a level of nitrate.

In yet another aspect, the invention relates to a method of manufacturing a nitrate measurement apparatus by providing a sample inlet for receiving a sample of gas, coupling a collection body to the sample inlet, disposing a filter within the body, coupling a heater to the body, coupling a gas inlet to the body, and coupling an NOₓ detector to the body.

The method, in an embodiment, comprises disposing an NO₂ extractor between said sample inlet and said collection body, and may further include disposing a catalyst capable of reducing NO₂ to NO between said collection body and said NOₓ detector.

### BRIEF DESCRIPTION OF THE FIGURES

The following figures depict certain illustrative embodiments of the invention in which like reference numerals refer to like elements. These depicted embodiments are to be understood as illustrative of the invention and not as limiting in any way.

FIG. 1 depicts a system for measuring nitrate levels as described herein.

FIG. 2 illustrates the accuracy of a method for measuring nitrate levels as described herein.

FIG. 3 shows the effect of atmospheric conditions on the method described herein.

FIG. 4 demonstrates a method of distinguishing between nitrate and NO₂ in a sample of gas using the systems and methods described herein.

FIGS. 5A and B present nitrate measurement results obtained over a 72-hour period.

FIG. 6 illustrates another system for measuring nitrate levels as described herein.

### DETAILED DESCRIPTION OF THE ILLUSTRATED EMBODIMENTS

The description below pertains to several illustrative embodiments of the invention. Although many variations of the invention may be envisioned by one skilled in the art, such variations and improvements are intended to fall within the compass of this disclosure. Thus, the scope of the invention is not to be limited in any way by the disclosure below.

The systems and methods disclosed herein are useful for measuring nitrate levels, for example, in the atmosphere, and may be capable of performing sample collection and analysis within about ten minutes. Thus, variability of nitrate levels can be determined over relatively short intervals, e.g., for use in epidemiological studies, regulatory monitoring, or other research. Furthermore, the system can be assembled or manufactured using convenient, commercially available components.

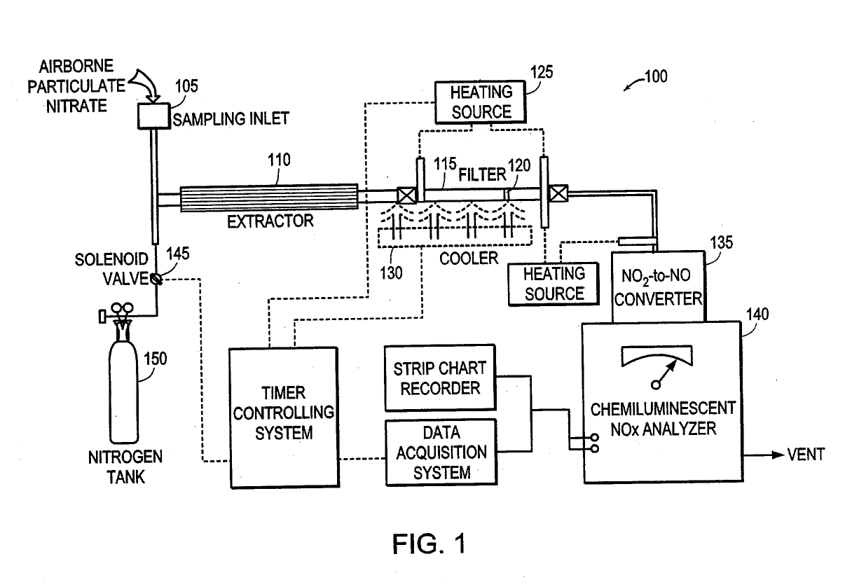

An exemplary system 100 for measuring nitrate levels is depicted in FIG. 1. The system 100 includes a sample inlet 105, an extractor 110, a collection body 115, a filter 120, a heater 125, a cooling system 130, a catalyst 135, a detector 140, a gas inlet 145, and a gas source 150. Other components, such as a control system, a data acquisition and recording system, or a second independent heater may optionally be included. Variations on the depicted system which are capable of functioning as described herein will be apparent to those of ordinary skill in the art and are intended to be encompassed by this disclosure.

A sample of gas, such as a sample of air or exhaust, may be received by the system using sample inlet 105. The sample of gas may be forced into the system 100, for example, by passing an exhaust stream through the system 100. Alternatively, the sample of gas may be drawn into the system 100 by a vacuum, e.g., by providing a vacuum beyond the detector 140, or by utilizing the Bernoulli effect, e.g., by passing a stream of gas rapidly past the inlet 105, e.g., using the gas inlet 145. The sample inlet 105 may include a selection platform for removing particles larger than about 2.5 microns, such as an inertial impactor or a filter, as is well known in the art. The sample of gas may then pass into the extractor 110 to remove contaminant gases. The extractor 110 may be a denuder, such as the honeycomb denuder described in U.S. Pat. No. 5,302,191 or an annular denuder, another diffusion denuder, or any other system known in the art for removing gases from a sample of gas. For example, the extractor 110 may include an acidic material, such as citric acid or sulfuric acid, to trap basic compounds, such as ammonia. In one embodiment, the extractor 110 is selected to remove at least 50%, or at least 90%, or even at least 95% of the gaseous NO₂ from the sample of gas, as gaseous NO₂ may introduce error into the nitrate measurement. Such an extractor may include a hydroxyl-bearing solvent, such as ethylene glycol, propylene glycol, glycerol, benzyl alcohol, or another hydroxylic solvent, and a base, including an inorganic base, such as a metal carbonate, bicarbonate, hydroxide, or phosphate, e.g., sodium hydroxide or potassium carbonate, and/or an organic base, such as an amine, e.g., 1,8-bis(dimethylamino)-naphthalene, diazabicyclooctane, diazabicyclononane, triethanolamine, diethanolamine, N,N-dimethyl-2-hydroxymethylaniline, or another organic base. In certain embodiments, the hydroxyl-bearing solvent and the organic base are selected to have low vapor pressures at atmospheric pressure, e.g., less than 50 Torr, or less than 10 Torr. Other systems for removing NO₂ or other selected contaminants are known in the art, and may be used alone or in any combination to remove any such compounds from the sample of gas.

The sample of gas may then pass into the collection body 115 and through filter 120. The filter 120 may then trap nitrate particles, in addition to other particles of similar size, e.g., about 2.5 microns or less, while allowing gaseous compounds to pass through. The collection body may be composed of any material capable of withstanding sufficient heat to perform the method as described herein, such as metal, ceramic, glass, quartz, or other heat-resistant material. For example, the collection body may be composed of steel, molybdenum, or an alloy comprising either material. The filter may be composed of any suitable material, e.g., quartz fibers, glass fibers, metal, or other material capable of withstanding temperatures sufficient to volatilize the trapped particles. Additionally, the filter may be made from polytetrafluoroethylene (PTFE) fibers. A stream of gas substantially free of oxygen, e.g., including less than about 5% or less than about 1% oxygen, such as nitrogen gas, helium, or argon, may then be passed over the trapped particles. This procedure helps to reduce unwanted oxidation of ammonia or other low oxidation state nitrogen-containing compounds, such as ammonium sulfate, during heating. The gas may be introduced using gas inlet 145 from gas source 150.

The heater 125 may then heat the filter 120 or the collection body 115 to volatilize the trapped particles. The heater may perform this function by any means known in the art. For example, the heater 125 may generate heat itself, such as with a heating element, e.g., a nichrome wire or a heat lamp, used to heat the collection body or filter, or it may apply current to the filter 120 or the collection body 115 to heat that element by resistance, or it may heat the sample by any other means known in the art. In addition to heating the collection body 115 and/or the filter 120, the heater 125 or a second heater may heat all or a portion of the path between the collection body 115 and the catalyst 135. Upon volatilization, nitrate may be converted to species such as HNO₃, NO₂, and NO which are carried by the stream of gas to the catalyst 135.

In certain embodiments, for example, wherein an extractor 110 is not used to remove NO₂ from the gas sample, a portion of the gaseous NO₂ in the sample of gas may be adsorbed by material on the filter, such as soot or other particulate matter, rather than passing through the filter. Upon heating such NO₂ may be desorbed at a temperature below that at which nitrate begins to substantially volatilize. In such embodiments, it may be advantageous to heat the collection body 115 and filter 120 gradually in order to release this unwanted NO₂ prior to detection and measurement of the NOₓ species liberated by volatilization of nitrate. NOₓ, as used herein, refers generally to NO and NO₂. By this method, more accurate nitrate determinations may be measured. Rapid heating, however, may permit more rapid cycling between collection and analysis phases. Similarly, cooling system 130 may cool the collection body 115 and/or filter 120 by any means, such as by passing an unheated fluid, e.g., air or water, over the exterior surface of the apparatus, to further enable more rapid cycling between collection and analysis phases. Thus, the speed of heating may be adjusted to balance cycling time with measurement accuracy, depending on the needs of a particular situation and the relative importance of accounting for NO₂ in the sample of gas.

The catalyst 135 may be any material, such as a molybdenum or carbon converter, ferrous sulfate, or any other material capable of reducing NO₂ to NO, as is known in the art. In embodiments where a detector 140 is used which is capable of simultaneously detecting NO₂ and NO, a catalyst 135 need not be included in the system, and the stream of gas may flow directly from the collection body 115 and filter 120 to the detector 140.

The detector 140 may be any component capable of detecting the amount of NOₓ in the stream of gas. A number of methods are known for detecting NOₓ in flowing gas streams. Perhaps the most well known and widely used process involves instruments using the chemiluminescent reaction of nitric oxide (NO) and ozone. The process operates by the reaction of ozone and nitric oxide within a reaction chamber having a transmissive window, allowing light produced by the chemiluminescent reaction to be monitored by a detector. Typical components using this process may be found in U.S. Pat. Nos. 3,967,933 to Etess et al.; 4,236,895 to Stahl; 4,257,777 to Dymond; 4,315,753 to Bruckenstein et al.; 4,657,744 to Howard; 4,765,961 to Schiff; and 4,822,564 to Howard. The use of a chemiluminescent nitrogen oxide monitoring device in controlling a nitrogen oxide removal unit placed on the outlet of a boiler is shown in U.S. Pat. No. 4,188,190 to Muraki et al. Because these systems are typically not capable of detecting NO₂ in the gas stream, a catalyst 135 may be employed in conjunction with such a detector.

Another procedure involves the use of an infrared beam, detector, and a comparator chamber. In U.S. Pat. No. 4,647,777 to Meyer, a beam of infrared light is passed through a gas sample and into a selective infrared detector. The beam is split and one portion passes through a chamber containing a fluid that absorbs the spectral wavelengths of the selected gas. The two beams are compared and the difference between the two beams gives an indication of the amount of a selected gas in the sample.

A semiconductor NOₓ sensor is described in U.S. Pat. No. 5,863,503. The resistance of this sensor is altered by the absorption of NO and NO₂. Such a detector 140 may thus simultaneously measure NO and NO₂ levels, and therefore may function accurately in the absence of a catalyst 135.

One of the above detectors, or any other detector capable of measuring NO or NO₂ concentrations, may be employed as detector 140. In the case of a detector which is capable of detection NO₂ but not NO, it may be advantageous to oxidize NO in the stream of gas to NO₂, for example, using an ozone generator or other source of oxidant. The detector 140 may include or may be coupled to a processor, plotter, or other recording apparatus for displaying, recording, or storing data collected by the detector 140.

In certain embodiments, the filter 120 may be replaced by an inertial impactor, which is also known to be useful for collecting particulate matter from a stream of gas. In order to volatilize the collected sample, the inertial impactor may be heated directly, or indirectly, as described above for a filter embodiment. Otherwise, the system is analogous to the system described above. Thus, in one embodiment, an inertial impactor is used in a system as described above which uses an NO₂ extractor, such as a diffusion denuder, as described above.

A system 100 as described above may be manufactured by coupling a sample inlet to a collection body, disposing a filter in said collection body, coupling said collection body to an NOₓ detector, and coupling a gas inlet to said body. In certain embodiments, the method may further include coupling an extractor, such as an NO₂ extractor as described above, between said sample inlet and said filter. When the detector employed does not adequately detect NO₂, a catalyst may be disposed between said detector and said collection body to reduce NO₂ to NO. Alternatively, if the detector employed does not adequately detect NO, an oxidizer may be disposed between said detector and said collection body. The components included in such a system may be any of the components set forth above or components that function equivalently or analogously.

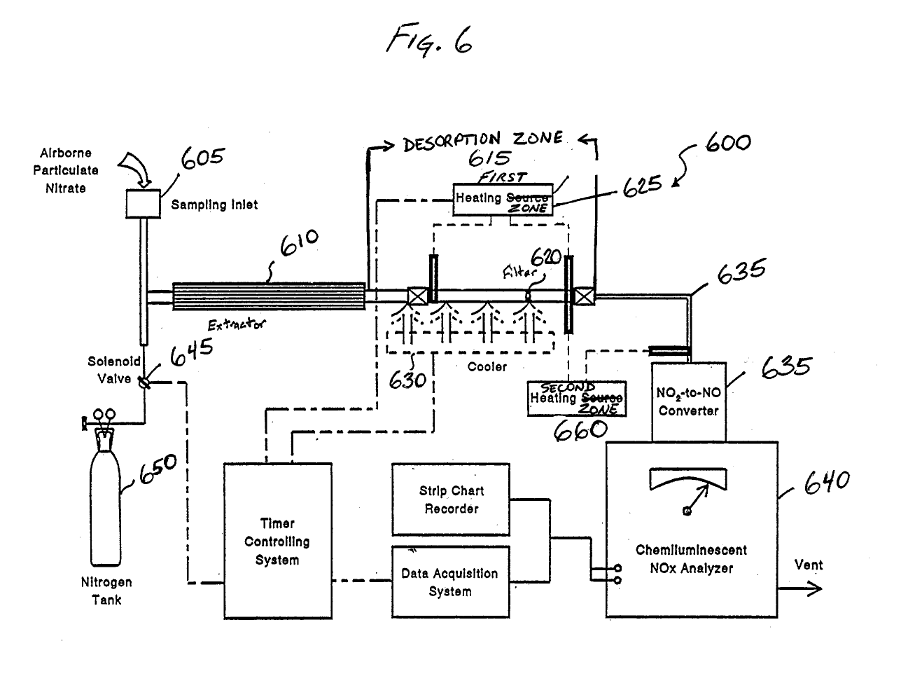

Referring now to FIG. 6, a system 600 is provided in accordance with another embodiment of the present invention for measuring nitrate. The system 600, in one embodiment, may include a sample inlet 605 to permit introduction of a sample of gas from which the nitrate level may be determined. The system 600 may also include a desorption zone 615 downstream from the sample inlet 605. Downstream in this context will mean distal to preceding structures within the system 600 and away from the point of gas introduction. Contained within the desorption zone 615 may be a filter 620 provided to trap particles, such as nitrate, present within the sample of gas. The system 600 further includes a first heating zone 625, coupled to the desorption zone 615, to heat the filter 620 and volatilize trapped particles within the filter 620. A gas inlet 645 may be provided to introduce a secondary flow of gas over the particles trapped by the filter 620 in order to reduce the possibility of unwanted oxidation of ammonia or other low oxidation state nitrogen containing compounds during heating. In one embodiment, the gas inlet 645 may be provided upstream from the desorption zone 615, between the filter 620 and the sample inlet 605, Upstream in this context will mean proximal to subsequent structures within the system 600 and towards the point of gas introduction. This secondary flow of gas may be substantially free of oxygen and be used to carry the volatized particles to an analyzer 640. The secondary flow of gas may be nitrogen supplied from a gas source 650.

Still referring to FIG. 6, the filter 620, used for trapping particles, such as nitrate, and located within the desorption zone 615, may be made, in an embodiment, from polytetrafluoroethylene (PTFE) fibers. PTFE is relatively inert and can reduce interferences from other gases which otherwise may be trapped by the filter. In addition, PTFE is believed not to trap, bind or react with nitrate, so as to enhance recovery efficiency. When using such PTFE filter 620, the first heating zone 625 may be maintained at a temperature which permits the filter 620 to be heated to range of from about 200° C. to 250° C. It should be noted that at this temperature range, although substantially all of the nitrate trapped within the filter 620 may be volatilized, not all nitric acid (HNO₃) may be converted to NO or NO₂. To that end, some of the nitric acid may be lost on the internal surfaces of the system 600, including the internal surfaces of pathway 635, as the stream of gas moves along the pathway 635 towards analyzer 640. Such a loss can affect the accuracy of the nitrate level measurement reading.

To minimize such a loss, still looking at FIG. 6, the system 600 may be provided with a second heating zone 660 to heat all or a portion of the pathway 635 between the desorption zone 615 and analyzer 640. This second heating zone 660 may heat the secondary gas stream, including the nitric acid and other volatilized particles in pathway 635 between the desorption zone 615 and the analyzer 640 to a temperature in a range of from about 600° C. to about 1000° C., to minimize deposition of volatized particles, such as nitric acid, on internal surfaces of the pathway 635. As a result, recovery efficiency can be kept relatively high. In addition, the second heating zone 600 may be used to convert nitric acid desorbed off the filter 620 to NO or NO₂. To provide the system 600 with the second heating zone 660, in one embodiment, nichrome wires may be wrapped around the pathway 635 and subsequently passing current through the wires to heat the pathway 635.

In accordance with another embodiment, the second heating zone 660 may be designed to heat the pathway 635 itself between the desorption zone 615 and the analyzer 640 up to about 100° C. to minimize the loss of particles, such as nitric acid, on the internal surfaces of the pathway 635 in this area. In this embodiment, the surfaces of pathway 635, rather than the gas stream in the pathway 635, may be heated to prevent volatilized particles from adhering to the internal surfaces.

Components of system 600 similar to like components described above in relation to system 100 may perform their functions or take the form of the system 100 embodiments described above. For example, the first heating zone 625 and second heating zone 660 may be similar or identical to the disclosed heater 125 embodiments as disclosed above. Similarly, the analyzer 640 of system 600 is correlative to the detector 140 of system 100, and thus, all embodiments described in relation to detector 140 are applicable as potential embodiments of analyzer 640. One skilled in the art will readily correlate like components between the two systems and understand the applicability of system 100 disclosures with respect to system 600.

In accordance with one embodiment, the section of the system 600 proximal or upstream to the desorption zone 615 can be maintained at or near ambient air temperature during the sample collection phase. This may be accomplished by using a sheath air source external to the system 600. By maintaining this section of the system 600 at ambient air temperature, premature volatilization of the nitrate within the gas sample during the sample collection phase may be minimized.

The system 600 detailed in FIG. 6 may also incorporate additional components as described in relation to the system 100 illustrated in FIG. 1 above. For example, the system 600 may include a selection platform coupled to the sample inlet 605 to remove particles larger than about 2.5 microns. Such a selection platform may be an inertial impactor, a filter, or any other suitable structure know in the art. Additionally, the system 600 may include an extractor 610 to remove contaminant gases contained within the sample gas. As described above in relation to system 100, such an extractor may be a denuder, an annular denuder, or any other system known in the art for removing contaminant gases from a gas sample.

The system 600 may further include a cooling system 630 to cool the desorption zone 615, and filter 620 therein, in order to adjust the speed of heating the desorption zone 615 and filter 620, thereby balancing the accuracy of nitrate measurements versus the speed of cycling time. In addition, the system 600 may incorporate a catalyst 635 capable of reducing NO₂ to NO such as a molybdenum or carbon converter, ferrous sulfate, or any other suitable material known in the art.

FIG. 6 may also be instructive in illustrating a variety of nitrate measuring methods. In accordance with one such method, a gas sample from which nitrate levels may be measured may first be introduced through a sample inlet 605. Next, particles, such as nitrate, contained within the gas sample may be trapped on a filter 620. Thereafter, a secondary stream of gas, which may be substantially free of oxygen, can be passed over the trapped particles. The filter 620 may then be heated to volatilize the trapped particles therein. The pathway 635 downstream from the filter 620 may also be heated to minimize volatilized particles, such as nitric acid, from depositing on internal surfaces of the pathway 635. Thereafter, the level of nitrate may be measured from the gas sample downstream from the filter 620.

It will also be apparent to one skilled in the art that additional methods related to those described above may be accomplished by incorporating disclosure described in relation to system 600 as well as system 100 above. For example, trapping particles may be accomplished by using a PTFE fiber filter 620. The filter 620 may thereafter be heated by the first heating zone 625 to a maximum of about 250° C., and NO₂ may be substantially removed from the gas sample prior to trapping particles. It will be further understood that this list is not inclusive, and numerous additional methods may be accomplished by incorporating the above disclosure.

The following examples are provided solely to further illustrate the nature and advantages of one embodiment of the present invention and are not intended to limit the scope of the invention in any way.

### Exemplification

A system as described above and depicted in FIG. 1 was tested to determine the accuracy and utility of the measurements recorded thereby.

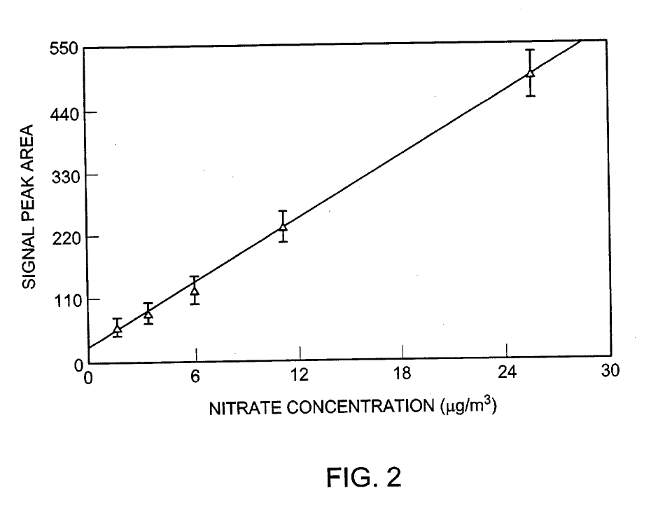

FIG. 2 shows that as nitrate concentration in the gas sample increases, instrument response increases in turn. Furthermore, the very linear fit indicates that the instrument provides a linear response and should measure nitrate levels accurately over a broad range of concentrations.

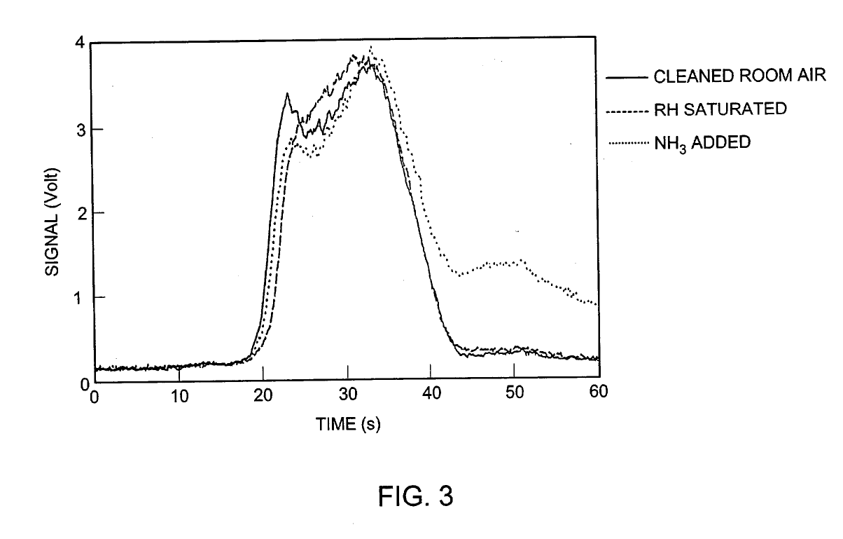

FIG. 3 shows that the introduction of species, such as water (relative humidity (RH) saturated) or ammonia, into the sample of gas does not significantly affect the nitrate level readings of the instrument. In all cases, the peak area is relatively similar.

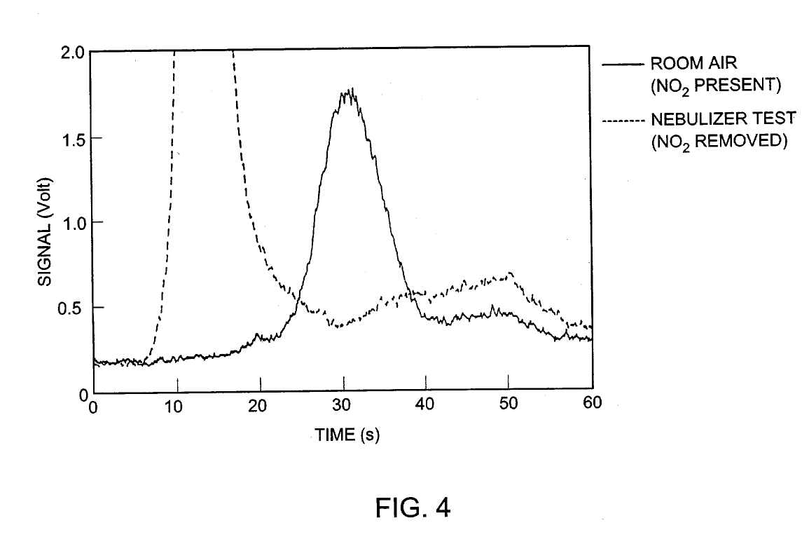

FIG. 4 illustrates how NO₂ in the gas sample as collected and NO₂ released from volatilization of nitrate particles can be distinguished using the present method, even in the absence of an extractor.

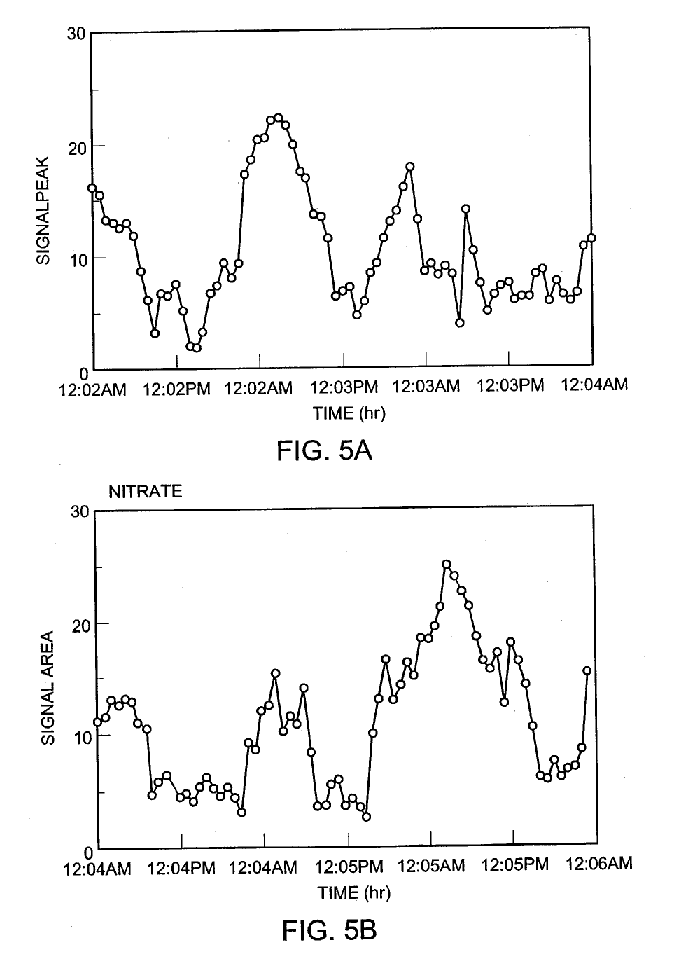

FIGS. 5A and SB present data collected from atmospheric air samples over three-day periods. Considerable variation can be seen within a given 24-hour period, and these variations can be elucidated because of the relatively short collection-analysis cycles possible using the systems and methods disclosed above.

All articles, patents, and other references set forth above are hereby incorporated by reference. While the invention has been disclosed in connection with the embodiments shown and described in detail, various equivalents, modifications, and improvements will be apparent to one of ordinary skill in the art from the above description. Such equivalents, modifications, and improvements are intended to be encompassed by the following claims.

What is claimed is:

1. A system for measuring nitrate levels, comprising:
a sample inlet to permit introduction of a gas sample from which nitrate levels may be measured;
a filter, downstream from the sample inlet, to trap particles from the gas sample;
a first heating zone to heat the filter and volatilize the trapped particles,
a second inlet, to introduce a secondary stream of gas to carry the volatilized particles downstream;
a second heating zone to heat a pathway through which the volatilized particles travel to minimize deposition of the volatized particles on internal surfaces of the pathway; and
an analyzer, downstream from the second heating zone to measure nitrate levels present in the secondary stream of gas.

2. The system of claim 1, wherein the volatilized trapped particles includes nitric acid, NO₂ and NO.

3. The system of claim 1, wherein the filter comprises polytetrafluoroethylene (PTFE) fibers.

4. The system of claim 1, further comprising an air source to maintain a sheath of air over a section of the system proximal to the filter at or near ambient air temperature.

5. The system of claim 1, wherein the second heating zone can generate temperatures higher than those generated by the first heating zone.

6. The system of claim 3, wherein the first heating zone can generate a temperature range of from about 200° C. to about 250° C.

7. The system of claim 1, wherein the second heating zone is designed to heat the surfaces of the pathway through which the volatilized particles travel to a temperature of about 100° C.

8. The system of claim 1, wherein the second heating zone comprises heating the pathway distal to the filter to a maximum of about 100° C.

9. The system of claim 1, wherein the secondary stream of gas includes gas substantially free of oxygen.

10. The system of claim 9, wherein the gas in the secondary stream is nitrogen.

11. The system of claim 1, further comprising a cooling system to adjust the speed of heating the filter.

12. A method for measuring nitrate levels, comprising:
providing a gas sample from which nitrate levels may be measured;
trapping particles present in the gas sample on a filter;
heating the filter to volatilize the trapped particles;
passing a secondary stream of gas through the filter to carry the volatilized particles downstream;
heating the secondary stream of gas downstream of the filter to minimize deposition of the volatilized particles on internal surfaces of a pathway along which the stream of gas is traveling; and
measuring a level of nitrate in the secondary stream of gas.

13. The method of claim 12, wherein in the step of trapping particles the filter includes providing a polytetrafluoroethylene (PTFE) fiber filter.

14. The method of claim 13, wherein heating the filter includes raising the temperature of the filter to a temperature ranging from about 200° C. to about 250° C.

15. The method of claim 12, wherein heating the secondary stream of gas includes heating the stream of gas to a temperature ranging from about 600° C. to about 1000° C.

16. The method of claim 12, wherein heating the secondary stream of gas includes heating surfaces of the pathway to a temperature of about 100° C.

17. The method of claim 12, further comprising substantially removing NO₂ prior to trapping particles.

18. The method of claim 12, wherein passing a secondary stream of gas includes utilizing a gas substantially free of oxygen.

19. The method of claim 12, wherein passing a secondary stream of gas substantially free of oxygen includes utilizing nitrogen as the gas.

20. The method of claim 12, wherein measuring a level of nitrate includes adsorbing NOₓ on a conductive material.

21. A system for measuring nitrate levels, comprising:
means for introducing a gas sample from which nitrate levels may be measured;
means for trapping particles present in the gas sample;
means for heating the trapped particles to volatilize said trapped particles;
means for introducing a stream of gas to the volatilized particles to carry the volatilized particles downstream;
means for heating the stream of gas downstream in a pathway to minimize deposition of the volatilized particles on internal surfaces of the pathway; and
means for measuring a level of nitrate from the stream of gas.
